(19) United States
(12) Patent Application Publication
(10) Pub. No.: US 2016/0327810 A1
(43) Pub. Date: Nov. 10, 2016

(54) LENS ASSEMBLY

(71) Applicant: ADAM PAUL CROSS, LAFAYETTE, LA (US)

(72) Inventor: ADAM PAUL CROSS, LAFAYETTE, LA (US)

(21) Appl. No.: 15/211,894

(22) Filed: Jul. 15, 2016

Related U.S. Application Data
(63) Continuation-in-part of application No. 14/554,754, filed on Nov. 26, 2014, now abandoned.

(51) Int. Cl. G02C 9/00; A63B 33/00; B63C 11/12; A61F 9/02

(52) U.S. Cl. CPC G02C 9/00; A61F 9/025; A63B 33/002; B63C 11/12; A61F 9/023; G02C 2200/08; G02C 2200/06

(57) ABSTRACT

A lens assembly provides an interchangeably removable frame and lens for a face mask for use in adjusting to a variety of different conditions that a user may encounter. The lens assembly beneficially provides a user with an option to interchangeably attach and detach a different lens to a face mask for a plurality of different conditions, including, but not limited to, a variety of visual conditions, a variety of eyesight accommodations, or a preferred design aesthetic. The lens assembly also beneficially provides for an easier and more efficient removal and replacement of the lens from the mask, without a user having to remove the mask from the user's face.

### CROSS REFERENCES TO RELATED APPLICATION

This application is a continuation-in-part of application Ser. No. 14/554,754, filed Nov. 26, 2014, incorporated herein by reference, currently pending.

### STATEMENTS AS TO THE RIGHTS TO THE INVENTION MADE UNDER FEDERALLY SPONSORED RESEARCH AND DEVELOPMENT

None

### BACKGROUND OF THE INVENTION

1. Field of the Invention

The present invention pertains to a face mask lens assembly for use in outdoor activities, including, but not limited to, underwater diving and snorkeling. More particularly, the present invention pertains to a replaceable lens apparatus for a face mask that can be quickly and efficiently changed to accommodate a variety of different conditions, including, but not limited to, visual conditions, eyesight, aesthetics or design preference.

2. Brief Description of the Prior Art

Generally, existing face masks lack the ability to adjust to a variety of different environmental conditions and/or visual needs of a user. A conventional face mask generally has a single, durable lens plate that is located directly in front of a user's eyes, with a substantially flexible casing (such as, for example, rubber, plastic, silicone, or any other similar material with like properties) that surrounds the lens plate and fits against a user's face. In most instances, the casing is designed to prevent water or debris from entering the space between the lens plate and the user's eyes, and thus, affecting a user's vision.

Additionally, a conventional face mask lacks the ability to easily and efficiently adjust to a variety of different conditions and/or visual needs of a user. For example, if a user needs a particular prescription lens, conventional face masks typically require permanent alteration to accommodate said prescription lens. Also, if a user needs special color filtration lenses to obtain optimum clarity for various environmental or underwater conditions, a user will generally require several different masks, each having a different color filtration lens in order to accommodate said various conditions.

As a result, conventional face masks generally require users to possess and maintain a single mask with a specific lens and design, or multiple different masks in order to accommodate all possible eyesight needs, visual conditions and/or aesthetic preferences. Purchasing and maintaining multiple different masks can be relatively expensive and inconvenient and can result in difficulties if a user is unsure of potential environmental and visual conditions that he or she may encounter, particularly when traveling within a natural body of water. Generally, in scuba diving, visual conditions are relative to the depth and color of the water, such as, for example, the difference between the light spectrum filtration that occurs at five (5) feet versus fifty (50) feet. The visual conditions encountered are also effected by the variety of color changes that are caused by a high presence of salinity, algae, and/or various soil types when suspended in a natural body of water.

As a result, there is a need for a face mask having a plurality of selectively removable and interchangeable lens assemblies, thereby accommodating a wide variety of possible eyesight needs, visual conditions and/or aesthetic preferences. The face mask should permit a user to quickly and efficiently remove and replace said lens assembly. Further, the removable and interchangeable lens assemblies can be capable of being retrofit to attach to and work with existing conventional face mask devices.

### SUMMARY OF THE INVENTION

The present invention comprises an interchangeable lens attachment, or lens assembly, that can selectively attach to a face mask, including, but not limited to, a traditional diving mask, a snorkeling mask, a full-face snorkeling mask, a full-face diving mask, swimming goggles, a commercial diving helmet, or a recreational diving helmet. Said attachable lens assembly can be easily and conveniently added to or removed from a supportive face mask without affecting any basic functionality of said face mask. Thus, said lens attachment is not necessary to create a water-tight seal between said lens attachment and a user because the conventional face mask that said lens attachment affixes to beneficially comprises a plurality of components that are necessary for creating a water-tight seal between said face mask and the user.

In a preferred embodiment, the lens attachment of the present invention comprises an attachment lens frame that can hold and support a lens having desired properties. By way of illustration, but not limitation, said lens can comprise a number of different desired properties and lens types, such as, for example, a color correction lens, a vision correction lens, a printed lens, a printed pinhole lens, a holographic lens, a welding lens, a cutting lens, and/or a clear lens. Said attachment lens frame can be selectively and removably attached to and connected to a mask apparatus, which can be worn by a user. Additionally, said attachment lens frame can comprise a plurality of attachment points in order to attachably connect a variety of different items, including, but not limited to, cameras, lights, and other equipment.

In a preferred embodiment, said attachment lens frame of the present invention is removably fastened to said conventional mask by way of an attachment point that selectively locks said attachment lens frame (and any attached lens) into place against said mask. Additionally, said attachment lens frame of the present invention can be used either singularly or in combination with additional attachments, thereby providing a user with the ability to use a single lens or multiple lenses at once.

By way of illustration, but not limitation, said attachment point of said attachment lens frame comprises a protrusion, or latch tab, to secure and lock said attachment lens frame into a latch tab recess on said mask. However, when said latch tab is pulled in a slight outward direction, away from said attachment lens frame, said latch tab is removed from the latch tab recess, and thus, becomes detached from said mask, thereby permitting removal of said attachment lens frame (and any attached lens installed therein) from said mask; said attachment lens frame can then be quickly and conveniently replaced with another attachment lens frame holding a lens having desired characteristics.

It is to be observed that said attachment lens frame of the present invention can be integrally attached to a face mask. Alternatively, said attachment lens frame can be affixed to a conventional face mask apparatus; said attachment lens frame can then accommodate an additional attachable and removable lens frame of the present invention. In this manner, the interchangeable lens assemblies of the present invention can be retrofit to existing conventional face masks, thereby permitting the quick and efficient interchangeability of various lenses having desired characteristics.

The lens assembly of the present invention allows a user to selectively change lenses in various conditions, such as, for example, while under water, without losing a tight fit of a mask against the user's face during the removal or attachment process. In addition, the lens assembly of the present invention allows a user to remove and replace lenses without having to remove a mask from said user's face. Further, the lens assembly of the present invention allows a user to attach a lens attachment in order to add a variety of different desired features or to remove the lens attachment if the features are undesired.

The lens assembly of the present invention allows for a plurality of removable and interchangeable lens assemblies, thereby permitting a user to selectively switch lenses to meet a wide variety of possible eyesight needs, visual conditions and/or aesthetic preferences, such as, for example, adding the benefit of color correction, vision correction, decoration, customization, and eye protection from damaging effects of bright light that is generated by welding and cutting operations. Further, said lens assembly of the present invention permits a user to quickly and efficiently remove and replace said lenses, and permits a user to quickly and efficiently join said dive mask attachments to a variety of different storage devices in order to keep all of the component parts of the lens assembly attached to the diver while diving, or for safe storage after use of said lens assembly.

### BRIEF DESCRIPTION OF THE DRAWINGS/FIGURES

The foregoing summary, as well as any detailed description of the preferred embodiments, is better understood when read in conjunction with the drawings and figures contained herein. For the purpose of illustrating the invention, the drawings and figures show certain preferred embodiments. It is understood, however, that the invention is not limited to the specific methods and devices disclosed in such drawings or figures.

FIG. 1 depicts an exploded front perspective view of a preferred embodiment of a lens attachment assembly of the present invention in a disconnected configuration.

FIG. 2 depicts a side view of a preferred embodiment of an attachment lens frame of the present invention in a disconnected configuration from a mask member.

FIG. 3 depicts a side perspective view of a preferred embodiment of a lens attachment assembly of the present invention in a connected configuration.

FIG. 4 depicts an exploded front perspective view of a preferred embodiment of a mask assembly of the present invention in a disconnected configuration.

FIG. 5 depicts a front perspective view of a preferred embodiment of a lens attachment assembly of the present invention in a connected configuration.

FIG. 6 depicts an exploded front view of a preferred embodiment of an attachment lens and an attachment lens frame of the present invention.

FIG. 7 depicts a rear view of a preferred embodiment of an attachment lens and an attachment lens frame of the present invention.

FIG. 8 depicts a side perspective view of a preferred embodiment of an attachment lens and an attachment lens frame of the present invention.

FIG. 9 depicts a side view of a preferred embodiment of an attachment lens and an attachment lens frame of the present invention in a connected configuration.

FIG. 10 depicts a side perspective view of a preferred embodiment of a plurality of lens attachment assemblies of the present invention in a connected configuration.

FIG. 11 depicts a side view of an alternate embodiment of a lens attachment assembly of the present invention in a connected configuration.

FIG. 12 depicts a side view of an alternate embodiment of a lens attachment assembly of the present invention in a connected configuration.

### DETAILED DESCRIPTION OF A PREFERRED EMBODIMENT

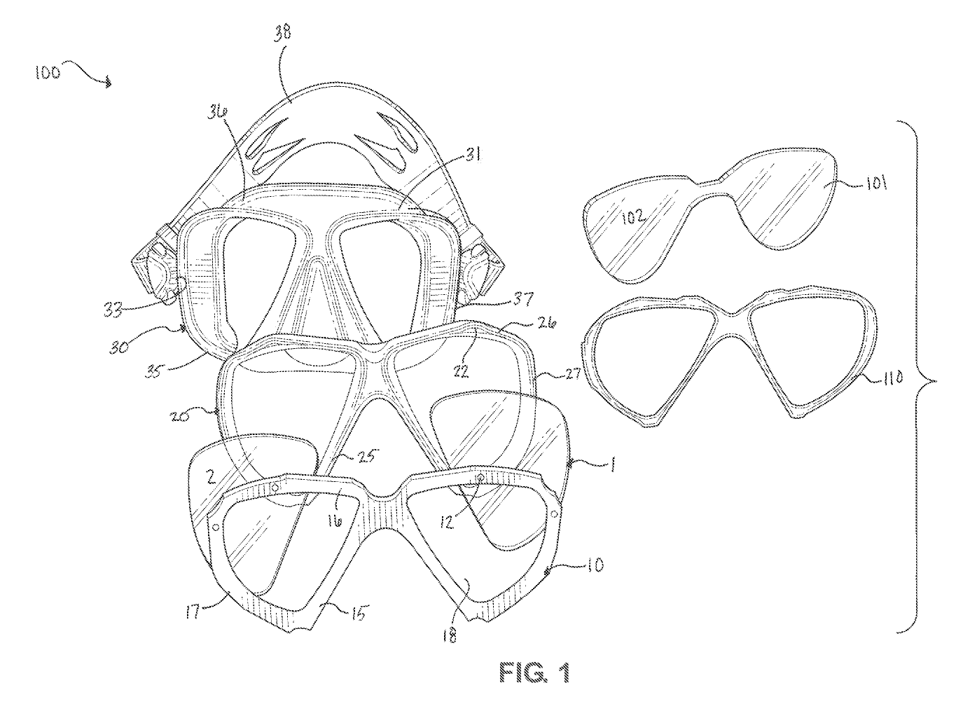

Referring to the drawings, FIG. 1 depicts an exploded front perspective view of a preferred embodiment of a lens attachment assembly 100 of the present invention generally comprising mask member 30, mask frame 20, lens frame 10 and lens 1 of the present invention in a disconnected configuration. Lens 1 comprises a substantially planar front surface 2 that has a relatively flat exterior in relation to a user's eyes and face when lens assembly 100 is being worn. In addition, lens 1 can be received within lens frame 10, thus allowing lens 1 to be semi-permanently joined and attached to lens frame 10 of the present invention. Further, although not illustrated in FIG. 1, in an alternate embodiment, lens frame 10 can be integrated with lens 1, thereby being constructed and manufactured from a single material that comprises both lens frame 10 and lens 1.

In a preferred embodiment, lens frame 10 generally comprises substantially curved base member 15, substantially curved top member 16, and substantially curved side members 17. Said base member 15 and top member 16 are oriented substantially parallel to each other, while said side members 17 are oriented substantially parallel to each other; said base member 15, top member 16 and side members 17 cooperate to form lens attachment frame defining inner opening 18. Said inner opening 18 comprises an orifice or aperture that, when worn, substantially surrounds a user's eyes, thereby allowing said user to visually see through lens assembly 100 of the present invention.

Lens frame 10 can beneficially comprise a plurality of attachment points 12 that correspond to a plurality of attachment points 112 of attachment lens frame 110 in a generally aligning orientation, thus allowing for a means of connection between said lens frame 10 and said attachment lens frame 110. The plurality of attachment points 12 are flush with a front surface of lens frame 10 and are generally used to house a plurality of magnets for use in temporarily attaching attachment lens frame 110 to lens frame 10. Attachment points 12 properly align with attachment points 112 of attachment lens frame 110, thereby beneficially creating a means of attachment for attachment lens frame 110 to lens frame 10 by use of a magnetic force, thus creating a unified assembly.

Lens frame 10 beneficially comprises a plurality of tabs 23 that can be received with tab recesses 22 of mask frame 20. Said cooperating tabs 23 and tab recesses 22 provide a means of attachment and connection for lens frame 10 to semi-permanently join and attach lens 1 to mask frame 20, and thus to mask member 30, by way of a clamping effect that is created by an attachment of lens frame 10 to mask frame 20.

Mask member 30 generally comprises a skirt member 31 having a substantially curved base member 35, a substantially curved top member 36, and substantially curved side members 37. Further, mask member 30 generally comprises mask frame 20 having a substantially curved base member 25, a substantially curved top member 26, and substantially curved side members 27 that substantially align and cooperate with base 35, top 36, and sides 37 of skirt member 31. Additionally, mask frame 20 substantially aligns and cooperates with base 15, top 16, and sides 17 of lens frame 10. In addition, although not illustrated in FIG. 1, mask frame 20 comprises a plurality of tab recesses 32 that are used as a means of attachment and connection points for lens frame 10 to semi-permanently join and attach to mask frame 20 of the present invention.

Additionally, although not depicted in FIG. 1, a strap member 38 can be beneficially located and connected to a mounting surface of sides of skirt member 31 through at least one strap attachment aperture 33, in order to provide a means to secure the lens assembly 100 around a user's head. Further, strap member 38 can also be beneficially located and connected to a surface of sides 27 of mask frame 20.

In an alternate embodiment, lens assembly 100 can comprise a “frameless” mask, wherein lens frame, mask frame, and skirt member can be manufactured and constructed from a single piece of relatively flexible material, such as, for example, silicone, or any other similar material exhibiting like characteristics. In an alternate embodiment, said frameless mask comprises a single component part, wherein lens 1 is permanently incased within said frameless mask.

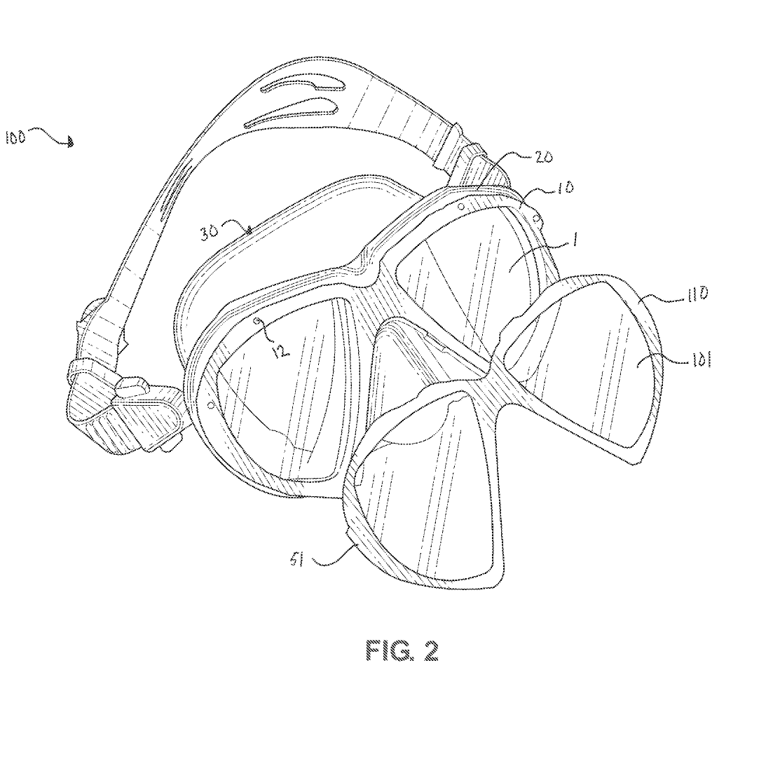

FIG. 2 depicts a side view of a preferred embodiment of attachment lens 101 semi-permanently connected to attachment lens frame 110, while disconnected from mask member 30. Thus, in a preferred embodiment, generally only attachment lens frame 110 and mask frame 20 are able to detachably interlock using locking assembly 50. This allows a user to conveniently latch and lock attachment lens frame 110 to mask frame 20 of mask member 30, while wearing mask member 30, and while wearing gloves, hand wear and/or other equipment in adverse water conditions, as opposed to attempting to replace a single lens or an entire mask. In this configuration, latch tab 51 is removed from latch tab recess 52. As depicted in FIG. 2, with said latch tab 51 no longer acting against the surfaces of latch tab recess 52, attachment lens frame 110 is no longer secured to lens frame 10 and, thus, said attachment lens frame 110 can be easily separated from lens frame 10.

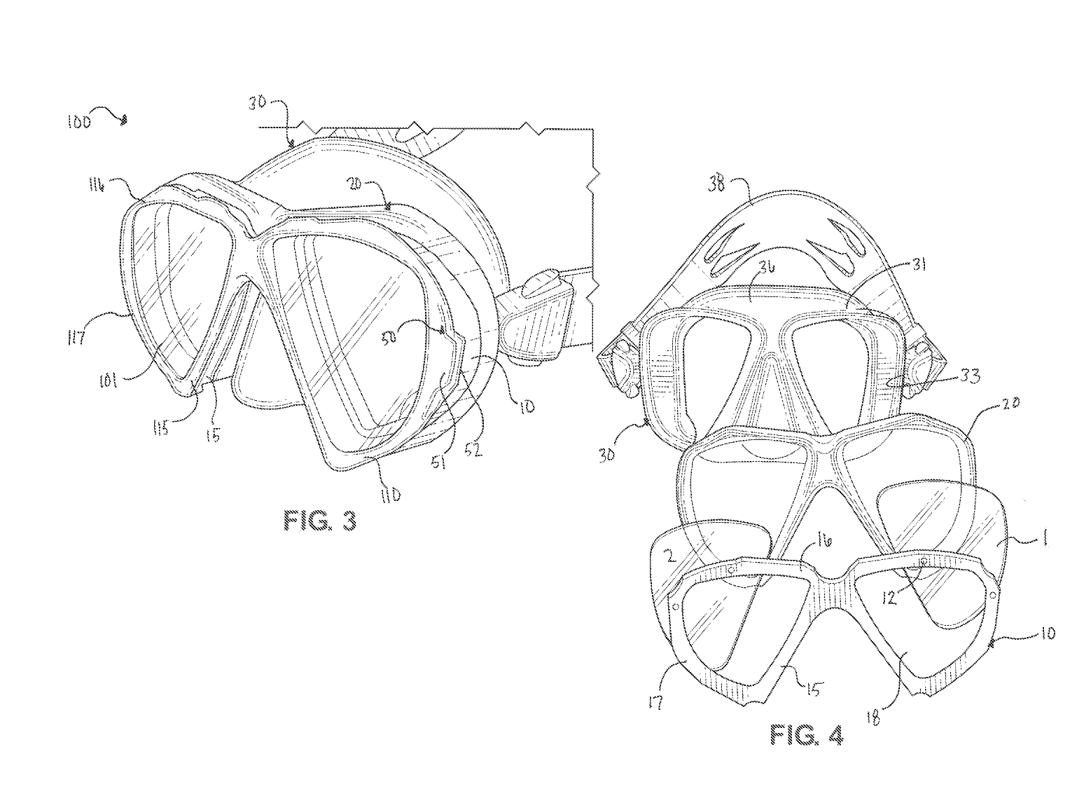

FIG. 3 depicts a side perspective view of a preferred embodiment of lens assembly 100 generally comprising mating assembly 50 in an adjoined position within attachment lens frame 110 and mask member 30, thereby allowing attachment lens frame 110 to attachably connect to mask member 30. As illustrated in FIG. 3, attachment lens frame 110 directly aligns and cooperates with said mask member 30. Thus, mating assemblies 50 of attachment lens member 110 directly align and correspond with guide tab recesses 52 of lens frame 10 of mask member 30. Guide tab recess 52 is used to help guide and position lens frame 10 and attachment lens frame 110 into place so that attachment points 112 and attachment points 12 can be more easily aligned, thereby securely connecting attachment lens frame 110 to lens frame 10.

In addition, base 115, top 116, and sides 117 of attachment lens frame 110 substantially align and cooperate with base 15, top 16, and sides 17 of lens frame 10 of mask member 30, thereby fastening together to form a cohesive unit. As a result, when coupled together, attachment lens frame 110 is properly guided and aligned with lens frame 10 of mask member 30 by way of mating assembly 50, thereby allowing attachment points 112 to interlock with attachment points 12.

Still referring to FIG. 3, in a preferred embodiment, a guiding mechanism or mating assembly 50 is located on a side of attachment lens frame 110 by way of a beneficially positioned protrusion, or latch tab 51, on said attachment lens frame 110; although not visible in FIG. 3, an opposing locking assembly is similarly mounted to attachment lens frame 110 on the opposite side of said attachment lens frame 110 from mating assembly 50. Latch tab 51 can be manufactured from a rigid material, such as, for example, a molded plastic material, or any other substantially similar structural material exhibiting desired characteristics, and extends in a relatively outward direction, generally away from attachment lens frame 110. Further, by way of illustration, but not limitation, mating assembly 50 can comprise a variety of different attachment means, including, but not limited to, magnets, snap clips, clip locks, holding clips, or spring clips.

As depicted in FIG. 3, guide tab recess 52 is located on a side of lens frame 10 at a corresponding position relative to latch tab 51 of mating assembly 50 on attachment lens frame 110. In this configuration, latch tab 51 acts against the surfaces of guide tab recess 52, thereby cooperating to securely lock attachment lens frame 110 against lens frame 10. In addition, although not visible in FIG. 3, it is to be observed that a guide tab recess 52 is also located on the opposite side of lens frame 10, in alignment with a latch tab 51 of mating member 50 of attachment lens frame 110.

In this manner, a plurality of mating assemblies can be mounted to attachment lens frame 110 in a corresponding relationship to a plurality of guide tab recesses of lens frame 10. Additionally, by way of illustration, but not limitation, lens frame 10 can comprise a variety of different attachment or connection points for mating assembly 50, including, but not limited to, a magnetic connection, a snap clip connection, a clip lock connection, a holding clip connection, or a spring clip connection.

FIG. 4 depicts an exploded front view of a preferred embodiment of mask member 30 of the present invention comprising lens 1, lens frame 10, mask frame 20, and skirt member 31. As depicted in FIG. 4, lens 1 comprises a substantially planar front surface 2 that has a relatively flat exterior in relation to a user's eyes and face when lens assembly 100 is being worn. In addition, lens 1 can be received within lens frame 10, thus allowing lens 1 to be semi-permanently joined and attached to lens frame 10 of the present invention. Further, although not illustrated in FIG. 4, in an alternate embodiment, lens frame 10 can be integrated with lens 1, thereby being constructed and manufactured from a single material that comprises both lens frame 10 and lens 1.

In a preferred embodiment, lens frame 10 generally comprises substantially curved base member 15, substantially curved top member 16, and substantially curved side members 17. Said base member 15 and top member 16 are oriented substantially parallel to each other, while said side members 17 are oriented substantially parallel to each other; said base member 15, top member 16 and side members 17 cooperate to form inner opening 18. Said inner opening 18 comprises an orifice or aperture that, when worn, substantially surrounds a user's eyes, thereby allowing said user to visually see through lens assembly 100 of the present invention.

Further, lens frame 10 can beneficially comprise attachment points 12 that correspond to a plurality of attachment points 112 of attachment lens frame 110 in a generally aligning orientation, thus allowing for a means of connection between said lens frame 10 and said attachment lens frame 110 (although not depicted in FIG. 4). Attachment points 12 are flush with a front surface of lens frame 10 and are generally used to house a plurality of magnets for use in temporarily attaching attachment lens frame 110 to lens frame 10. Attachment points 12 properly align with attachment points 112 of attachment lens frame 110, thereby beneficially creating a means of attachment for attachment lens frame 110 to lens frame 10 by use of a magnetic force, thus creating a unified assembly.

Although not illustrated in FIG. 4, lens frame 10 of mask member 30 comprises a plurality of guide tab recesses 52 that are used as a means of guiding and properly aligning attachment lens frame 110 in order to semi-permanently join and attach attachment lens frame 100 to lens frame 10 of mask frame 30 of the present invention. The plurality of latching tabs 51 of attachment lens frame 110 extend in a relatively outward direction from attachment lens frame 110, and can be received within guide tab recesses 52 of lens frame 10 of mask frame 30. Further, strap member 38 can be beneficially located and connected to a mounting surface of sides of skirt member 31 through at least one strap attachment aperture 33, in order to provide a means to secure mask member 30, and ultimately lens assembly 100, around a user's head.

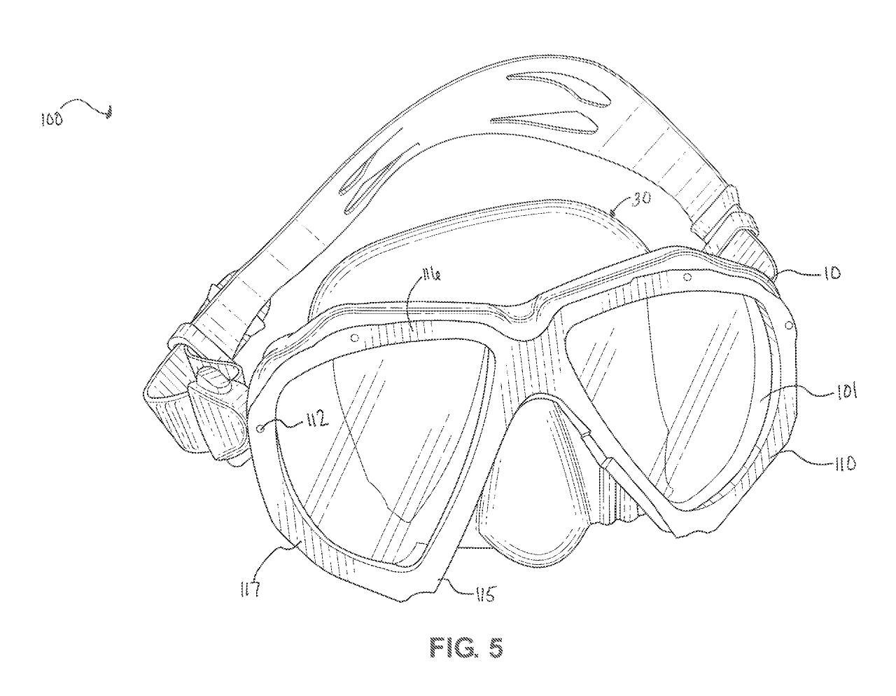

FIG. 5 depicts a front view of a preferred embodiment of lens assembly 100 of the present invention generally comprising attachment lens 101 semi-permanently connected to attachment lens frame 110, which is semi-permanently connected to mask member 30. As illustrated in FIG. 5, attachment lens frame 110 directly aligns and cooperates with lens frame 10 of mask member 30. Thus, mating assemblies 50 of attachment lens frame 110 directly align and correspond with guide tab recesses 52 of lens frame 10 of mask member 30, thereby guiding and properly aligning attachment lens frame 110 to lens frame 10. Once properly aligned, attachment points 112 are then properly aligned and connected with attachment points 12, thereby allowing attachment lens frame 110 to securely join to lens frame 10. In addition, base 115, top 116, and sides 117 of attachment lens frame 110 substantially align and cooperate with base 15, top 16, and sides 17 of lens frame 10 of mask member 30, thereby fastening together to form a cohesive unit.

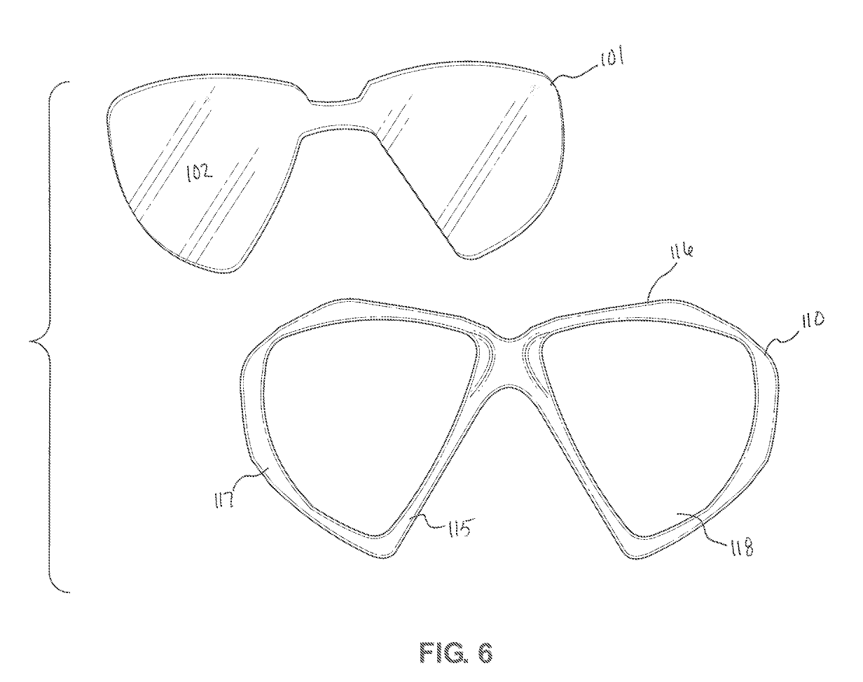

FIG. 6 depicts a front view of a preferred embodiment of attachment lens 101 of the present invention in a disconnected configuration from attachment lens frame 110. Attachment lens 101 comprises a substantially planar front surface 102 that has a relatively flat exterior in relation to a user's eyes and face when lens assembly 100 is being worn. In addition, although not depicted in FIG. 6, attachment lens 101 further comprises a plurality of attachment points 112 that correspond to a plurality of attachment points 12 of lens frame 10 in a generally aligning orientation, thus allowing for a means of connection between said lens frame 10 and said attachment lens frame 110. Attachment points 112 are flush with a front surface of attachment lens frame 110 and are generally used to house a plurality of magnets for use in temporarily attaching attachment lens frame 110 to lens frame 10. Attachment points 112 properly align with attachment points 12 of lens frame 10, thereby beneficially creating a means of attachment for attachment lens frame 110 to lens frame 10 by use of a magnetic force, thus creating a unified assembly.

Still referring to FIG. 6, in a preferred embodiment, attachment lens frame 110 generally comprises base member 115, top member 116, and side members 117, wherein base member 115 and top member 116 are oriented substantially parallel to each other, while side members 117 are oriented substantially parallel to each other; said base member 115, top member 116 and side members 117 cooperate to form attachment lens frame 110 defining inner opening 118. Inner opening 118 comprises an orifice or aperture that, when worn, substantially surrounds a user's eyes, thereby allowing said user to visually see through lens assembly 100 of the present invention.

Attachment lens 101 can be beneficially secured to attachment lens frame 110 by way of a variety of different attachment means, including, but not limited to, molecular bondage, adhesions, mechanical fasteners, or any other secure attachment means. Moreover, although not illustrated in FIG. 6, in an alternate embodiment, attachment lens frame 110 can be completely integrated with attachment lens 1, thereby being constructed and manufactured from a single material that comprises both attachment lens frame 110 and attachment lens 1.

Additionally, in an alternate embodiment, attachment lens frame 110 can comprise a plurality of attachment points on an outer surface of said attachment lens frame 110 in order to attachably connect a variety of different items, including, but not limited to, cameras, lights, lens holders, dive mask covers, or any other similar equipment. Said alternate equipment can be securely fastened to said attachment lens frame 110 by way of a plurality of magnets, or any other similar fastening means, in order to create a temporary mount for mounting said alternate items and equipment.

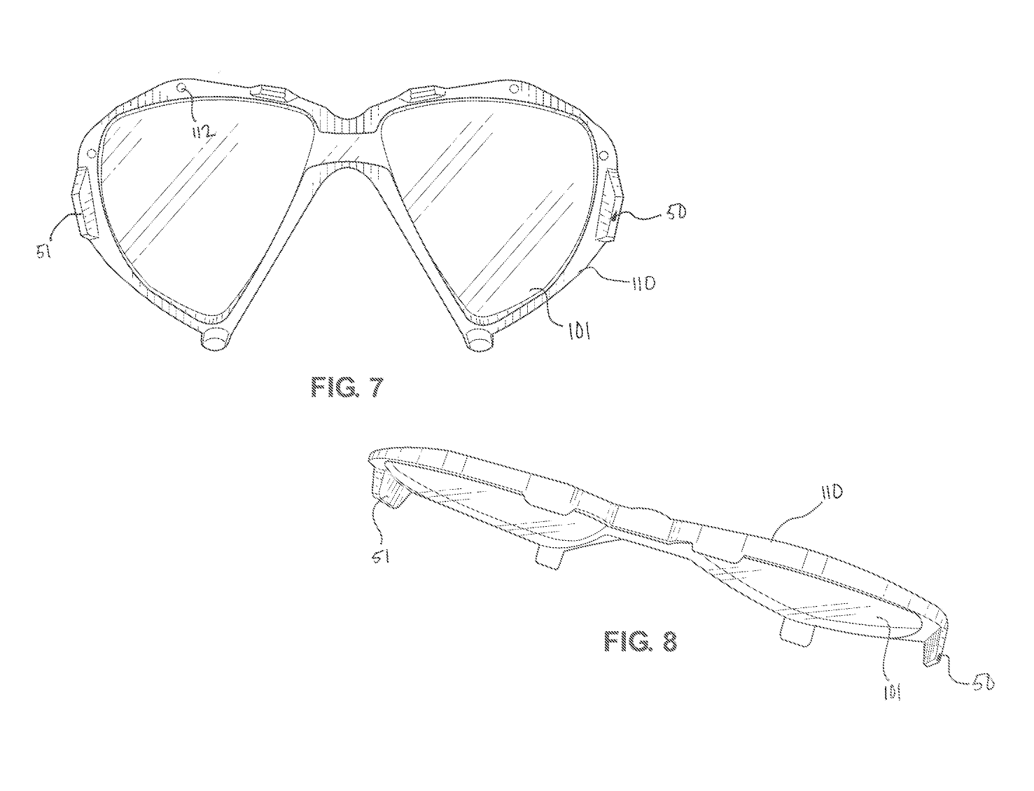

FIG. 7 depicts a rear view of a preferred embodiment of attachment lens 101 and attachment lens frame 110 of the present invention in a semi-permanently connected configuration. Attachment lens frame 110 comprises guiding mechanism or mating assembly 50 that is located on a side of attachment lens frame 110 by way of a plurality of beneficially positioned protrusions, or latch tabs 51, on said attachment lens frame 110. Latch tab 51 can be manufactured from a rigid material, such as, for example, a molded plastic material, or any other substantially similar structural material exhibiting desired characteristics, and extends in a relatively outward direction, generally away from attachment lens frame 110. Further, by way of illustration, but not limitation, mating mechanism 50 can comprise a variety of different attachment and connection means, including, but not limited to, magnets, snap clips, clip locks, holding clips, or spring clips.

FIG. 8 depicts a rear perspective view of a preferred embodiment of attachment lens 101 and attachment lens frame 110 of the present invention in a semi-permanently connected configuration generally comprising a plurality of latch tabs 51 for use in guiding and properly aligning attachment lens frame 110 to lens frame 10 of mask member 30 or to additional attachment lens frames 210 in order to use multiple lenses at a single time. Further, latch tabs 51 comprise a substantially indented configuration in order to assist a user in guiding attachment lens 101 and attachment lens frame 110 into a desired attachable position.

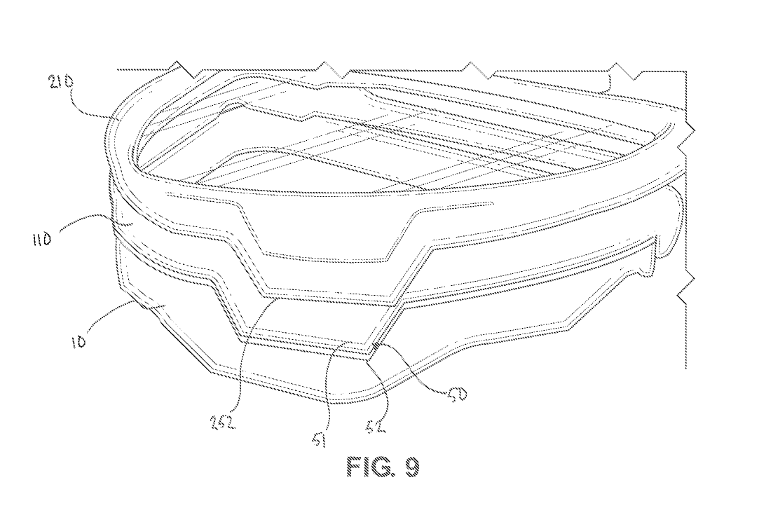

FIG. 9 depicts a side view of a preferred embodiment of attachment lens frame 110 of the present invention semi-permanently connected to multiple attachment lens frames. As depicted in FIG. 9, attachment lens frame 110 with attachment lens 101 directly aligns and cooperates with lens frame 10 of mask member 30 or with an additional attachment lens frame 210. Thus, mating assemblies 50 of attachment lens frame 110 directly align and correspond with guide tab recesses 52 of lens frame 10 of mask member 30 or with guide tab recesses 252 of an additional attachment lens frame 210.

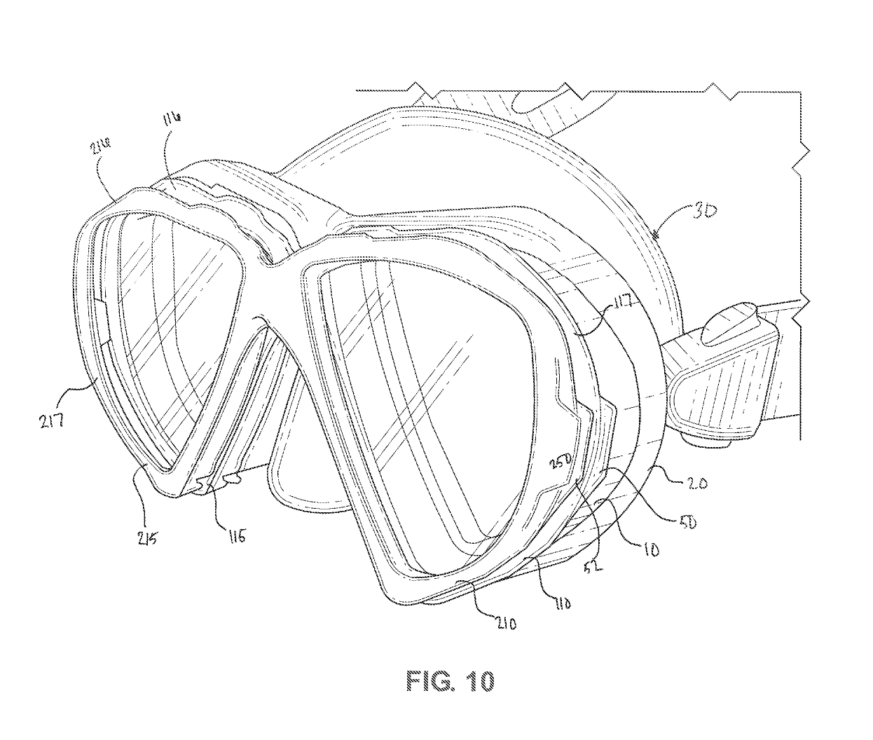

FIG. 10 depicts a side perspective view of a preferred embodiment of lens assembly 100 of the present invention comprising multiple attachment lens frames semi-permanently connected to attachment lens frame 110. As illustrated in FIG. 10, additional attachment lens frame 210 directly aligns and cooperates with attachment lens frame 110, which directly aligns and cooperates with said mask member 30. When additional attachment lens frame 210 cooperates with attachment lens frame 110, mating assembly 50 of attachment lens frame 100 is in an adjoined position with additional attachment lens frame 210, thereby attachably connecting and securing additional attachment lens frame 210 to attachment lens frame 110. Thus, mating assemblies 250 of additional attachment lens member 210 directly align and correspond with guide tab recesses 52 of attachment lens frame 110.

In addition, a base 215, a top 216, and sides 217 of additional attachment lens frame 210 substantially align and cooperate with base 115, top 116, and sides 117 of attachment lens frame 110, thereby fastening together to form a cohesive unit. As a result, when coupled together, additional attachment lens frame 210 and attachment lens frame 110 are properly aligned by way of mating assembly 250.

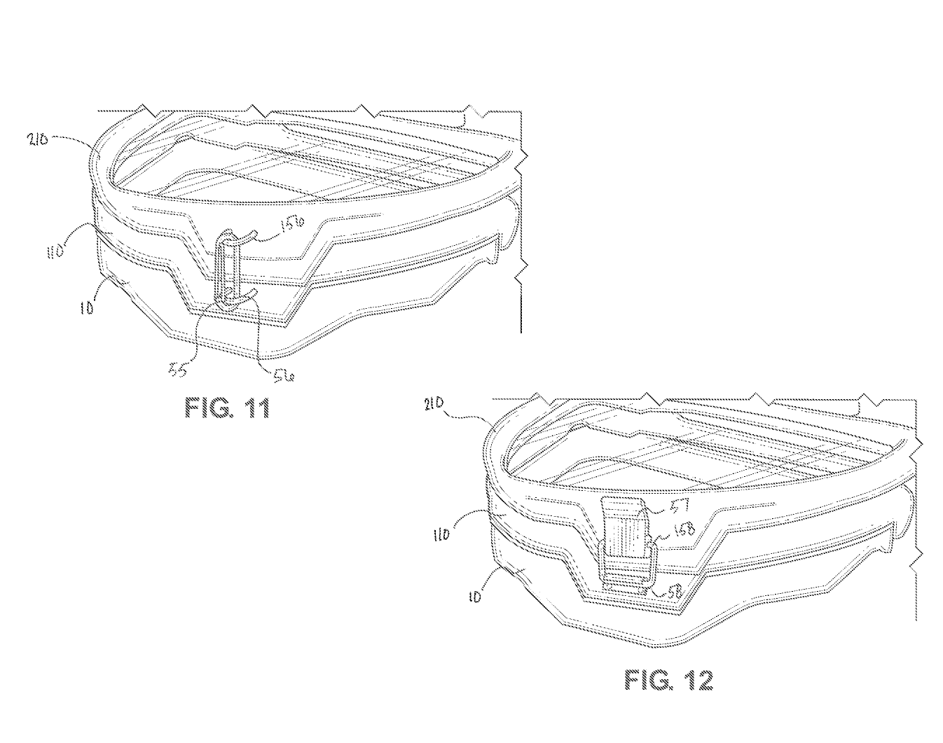

FIG. 11 depicts a side view of an alternate embodiment of lens assembly 100 of the present invention comprising multiple attachment lens frames semi-permanently connected to attachment lens frame 110. As depicted in FIG. 11, attachment lens frame 110 with attachment lens 101 directly aligns and cooperates with lens frame 10 of mask member 30 or with an additional attachment lens frame 210. Further, in an alternate embodiment, snap clip 55 of attachment lens frame 110 directly aligns and corresponds with snap clip connection 56 of lens frame 10 of mask member 30; alternatively, snap clip 255 of additional attachment lens frame 210 can directly align and correspond with snap clip connection 156 of attachment lens frame 110. In this manner, a plurality of locking assemblies can be mounted to additional attachment lens frame 210 in a corresponding relationship to a plurality of snap clip connections of attachment lens frame 110, or a plurality of locking assemblies can be mounted to attachment lens frame 110 in a corresponding relationship to a plurality of snap clip connections of lens frame 10.

FIG. 12 depicts a side view of an alternate embodiment of lens assembly 100 of the present invention comprising multiple attachment lens frames semi-permanently connected to attachment lens frame 110. As depicted in FIG. 12, attachment lens frame 110 with attachment lens 101 directly aligns and cooperates with lens frame 10 of mask member 30 or with an additional attachment lens frame 210. Further, in an alternate embodiment, toggle clip 57 of attachment lens frame 110 directly aligns and corresponds with toggle clip connection 58 of lens frame 10 of mask member 30; alternatively, toggle clip 257 of additional attachment lens frame 210 can directly align and correspond with toggle clip connection 158 of attachment lens frame 110. In this manner, a plurality of locking assemblies can be mounted to additional attachment lens frame 210 in a corresponding relationship to a plurality of toggle clip connections of attachment lens frame 110, or a plurality of locking assemblies can be mounted to attachment lens frame 110 in a corresponding relationship to a plurality of toggle clip connections of lens frame 10.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. An interchangeable lens assembly for a face mask comprising:
a. a mask adapted to be worn over a face;
b. a lens frame removably attached to said mask; and
c. a lens disposed in said lens frame.

2. The interchangeable lens assembly of claim 1, further comprising a latching mechanism adapted to removably attach said lens frame to said mask.

3. The interchangeable lens assembly of claim 2, wherein said latching mechanism further comprises:
a. a locking tab member disposed in a relatively outward direction from said lens frame; and
b. a clasp member defining a recess in said mask for receiving said locking tab member.

4. The interchangeable lens assembly of claim 3, wherein said latching mechanism comprises a magnetic connection.

5. The interchangeable lens assembly of claim 3, wherein said latching mechanism comprises a snap clip connection.

6. The interchangeable lens assembly of claim 3, wherein said latching mechanism comprises a toggle clip connection.

7. The interchangeable lens assembly of claim 3, wherein said latching mechanism comprises a holding clip connection.

8. The interchangeable lens assembly of claim 3, wherein said latching mechanism comprises a spring clip connection.

9. The interchangeable lens assembly of claim 1, further comprising an attachment mechanism disposed on an outer surface of lens frame adapted to removably attach a plurality of connection devices.

10. The interchangeable lens assembly of claim 1, further comprising:
a. a second lens frame, removable attachable to said lens frame;
b. a second lens disposed in said second lens frame.

11. The interchangeable lens assembly for a face mask comprising:
a. a mask adapted to be worn over a face;
b. a lens frame removable attached to said mask;
c. a lens disposed in said lens frame;
d. a second lens frame removably attached to said lens frame; and
e. a second lens disposed in said second lens frame.

12. The interchangeable lens assembly of claim 11, further comprising a latching mechanism adapted to removably attach said lens frame to said mask.

13. The interchangeable lens assembly of claim 11, further comprising a latching mechanism adapted to removable attach said second lens frame to said lens frame.

14. The interchangeable lens assembly of claim 12, wherein said latching mechanism further comprises:
a. a locking tab member disposed in a relatively outward direction from said lens frame; and
b. a clasp member defining a recess in said mask for receiving said locking tab member.

15. The interchangeable lens assembly of claim 13, wherein said latching mechanism further comprises:
a. a locking tab member disposed in a relatively outward direction from said lens frame; and
b. a clasp member defining a recess in said mask for receiving said locking tab member.

16. The interchangeable lens assembly of claim 15, wherein said latching mechanism comprises a magnetic connection.

17. The interchangeable lens assembly of claim 15, wherein said latching mechanism comprises a snap clip connection.

18. The interchangeable lens assembly of claim 15, wherein said latching mechanism comprises a toggle clip connection.

19. The interchangeable lens assembly of claim 15, wherein said latching mechanism comprises a holding clip connection.

20. The interchangeable lens assembly of claim 15, wherein said latching mechanism comprises a spring clip connection.

21. The interchangeable lens assembly of claim 11, further comprising an attachment mechanism disposed on an outer surface of lens frame adapted to removably attach a plurality of connection devices.
